(19) United States
(12) Patent Application Publication
(10) Pub. No.: US 2016/0051499 A1
(43) Pub. Date: Feb. 25, 2016

(54) COMPARATIVE EFFICACY AND TOLERABILITY OF DAPSONE 5% IN ADULT VERSUS ADOLESCENT FEMALES WITH ACNE VULGARIS

(71) Applicant: Allergan, Inc., Irvine, CA (US)

(72) Inventors: Conor J. Gallagher, San Clemente, CA (US); Manher Joshi, Santa Ana, CA (US)

(21) Appl. No.: 14/815,665

(22) Filed: Jul. 31, 2015

Related U.S. Application Data
(60) Provisional application No. 62/031,498, filed on Jul. 31, 2014.

(51) Int. Cl. A61K 31/145; A61K 9/00

(52) U.S. Cl. CPC A61K 31/145; A61K 9/0014

(57) ABSTRACT

The present invention provides a method of treating facial acne vulgaris in an adult female (≧18 years of age) in need of such treatment, comprising topically administering dapsone 5% gel twice daily to the face of the adult female.

### CROSS-REFERENCE TO RELATED APPLICATIONS

This application claims the benefit of U.S. Provisional Application No. 62/031,498 filed on Jul. 31, 2014, the entire contents of which is incorporated herein by reference.

### FIELD OF THE INVENTION

The present invention is directed to a method of treating facial acne vulgaris in a subject by topical administration of dapsone 5% gel.

### BACKGROUND

Acne vulgaris (AV) in adult women has been receiving increased attention both in the United States and globally, as the frequency of office visits for AV affecting post-adolescent women appears to be increasing. For example, a survey of 1013 respondents in the US showed that 51%, 35%, and 26% of women report having AV in their 20s, 30s, and 40s, respectively.

Additionally, AV is common in adult women of all ethnicities, skin types (oily, dry, combination, sensitive), and skin colors (Fitzpatrick skin type I-VI). Both visibly non-inflammatory lesions (e.g. comedones) and visibly inflammatory lesions (e.g. papules, pustules) are found in both adolescent and adult females with AV, and the anatomic distribution of AV is similar overall in both subpopulations. Available data and clinical observation have shown that the relative quantities of facial AV lesion types overlap among patients in both age-related subsets. Although a “U-shaped pattern” of predominantly inflammatory papules involving the lower cheeks, jawline, and anterior and lateral neck has been noted in some adult women with AV, and it has been stated that AV in adult women presents predominantly as inflammatory lesions, it has been reported that approximately three-fourths of adult women with AV present with a mixed pattern of comedonal and inflammatory facial acne lesions.

Most of the discussions about treatment of AV in adult women focus on the use of systemic therapies such as oral contraceptives and spironolactone, with little emphasis on topical therapies. Overall, controlled trials evaluating the efficacy and safety of topical agents specifically in adult women with AV are lacking, with subgroup analyses completed with only a few therapeutic agents and formulations

### SUMMARY OF THE INVENTION

The present disclosure provides methods of treating facial acne vulgaris in an adult female (≧18 years of age) in need of such treatment, comprising topically administering dapsone 5% gel twice daily to the face of the adult female. In one embodiment, the treatment results in statistically greater significant reductions in non-inflammatory and total lesion counts in the adult female compared to adolescent females (aged 12-17 years), wherein the reduction in inflammatory lesion is statistically the same in both the adult and adolescent female. In another embodiment, the reduction in non-inflammatory lesion count is about 20-33% higher (e.g., 20% higher, 25% higher, 30% higher, or 33% higher) in the adult female compared to the reduction in the adolescent female. In another embodiment, the reduction in total lesion count is about 15-20% higher (e.g., 15% higher, 17% higher, or 20% higher) in the adult female compared to the reduction in the adolescent female. In another embodiment, the treatment is for a period of about 12 weeks.

The present disclosure also provides a method of reducing the non-inflammatory and total lesion counts associated with acne vulgaris in an adult female (≧18 years of age) comprising topically administering dapsone 5% gel twice daily to the face of the adult female. In another embodiment, the treatment results in a statistically significant reduction in inflammatory lesion count in the adult female that is statistically the same as the reduction in inflammatory lesion count in an adolescent (aged 12-17 years) female. In another embodiment, the reduction in non-inflammatory lesion count is about 20-33% higher (e.g., 20% higher, 25% higher, 30% higher, 33% higher) in the adult female compared to the reduction in the adolescent female. In another embodiment, the reduction in total lesion count is about 15-20% higher (e.g., 15% higher, 17% higher, 20% higher) in the adult female compared to the reduction in the adolescent female. In another embodiment, the treatment is for a period of about 12 weeks.

The present invention also provides a method of increasing the efficacy of dapsone 5% gel in treating acne vulgaris in a female population, comprising topically administering dapsone 5% gel twice daily to the face of adult (≧18 years of age) females, wherein said administering results in statistically greater significant reductions in non-inflammatory and total lesion counts in the adult females compared to adolescent (aged 12-17 years) females, thereby increasing the efficacy of dapsone 5% gel in treating acne vulgaris in the female population. In another embodiment, the reduction in inflammatory lesion count is statistically the same in both the adult and adolescent females. In another embodiment, the reduction in non-inflammatory lesion count is about 20-33% higher in the adult females compared to the reduction in the adolescent females. In another embodiment, the reduction in total lesion count is about 15-20% higher in the adult females compared to the reduction in the adolescent females. In another embodiment, the administering of dapsone 5% gel is for a period of about 12 weeks.

### BRIEF DESCRIPTION OF THE FIGURES

FIGS. 1A-1C show the efficacy of dapsone 5% gel in adolescent and adult women. FIG. 1(A) illustrates the GAAS rating at baseline and week 12; FIG. 1(B) shows the percentage of dapsone 5% gel-treated subjects achieving GASS success (score 0 or 1) at week 12. FIG. 1(C) illustrates the mean change from baseline in GAAS in dapsone 5% gel and vehicle gel-treated subjects. GAAS=Global Acne Assessment Scale. *p<0.001 vs baseline.

FIG. 2 shows the effect of Dapsone 5% gel on tolerability in adolescent and adult women with acne. BL=baseline

### DETAILED DESCRIPTION

Described herein are methods of treatment of acne vulgaris with a dapsone 5% gel. Methods of treatment of AV with dapsone 5% gel in specific patient populations, such as women, or more specifically adult women (age >18 years), are also disclosed. Methods of treatment of AV with dapsone 5% gel with increased efficacy in specific patient populations, such as adult women, as compared to adolescent women are also disclosed.

### Dapsone 5% Gel

The dapsone 5% gel may be the commercially available ACZONE, available from Allergan, Inc. ACZONE is covered by U.S. Pat. Nos. 5,863,560; 6,060,085; and 6,620,435, which are all incorporated herein by reference in their entirety for the purpose of describing dapsone gel formulations, methods for making the formulations, and methods of treatment using the formulations, and are considered to be a part of this specification. Dapsone 5% gel includes dapsone in an amount of 5% by weight (or 5 wt %) of the total dapsone gel formulation.

Dapsone 5% gel is a sulfone derivative that has been reported to demonstrate a variety of anti-inflammatory properties. When applied topically to the face twice daily (BID) it was found to be effective for AV over a duration at least 12 months. In two large phase III, double-blind, randomized, vehicle-controlled, 12-week trials in AV in subjects >12 years of age, dapsone 5% gel applied BID was found to be superior to vehicle gel in reducing inflammatory, non-inflammatory, and total lesions from baseline. The outcomes of these phase III studies led to the approval of dapsone 5% gel BID for AV by the United States (US) Food and Drug Administration (FDA) in 2005.

### Methods of Treatment

According to some embodiments, a dapsone 5% gel is used to treat acne vulgaris in an appropriate patient population. The method of treatment of acne vulgaris using dapsone 5% gel may be effective to treat female patients. The method of treatment of acne vulgaris using dapsone 5% gel may be effective to treat adult female patients, having an age greater than or equal to 18 years old. The method of treatment of acne vulgaris using dapsone 5% gel may be effective to treat adolescent female patients, having an age in the range of 12 years old to 17 years old. In some embodiments, method of treatment of acne vulgaris using dapsone 5% gel may be effective to treat adult female patients with greater efficacy than adolescent female patients having an age between 12 years old and 17 years old.

In some embodiments, the dapsone 5% gel is the only therapeutic treatment that could affect AV applied to the face of the patient according to the methods of treatment described herein. In some embodiments, no other systemic agents, immunosuppressive agent, or oral isotretinoin are used in the method of treatment.

In an embodiment, a patient having acne vulgaris can perform the method of treatment with a dapsone 5% gel at a sufficient frequency for a period of time effective to improve the acne vulgaris in a patient in need thereof. In some embodiments, the dapsone 5% gel can be administered to the skin of the face of the patient having acne vulgaris at a frequency of one a day. In some embodiments, the dapsone 5% gel can be administered to the skin of the face of the patient having acne vulgaris at a frequency of twice a day. When the dapsone 5% gel is administered once daily, it can be done at various times, e.g., nightly or in the morning. Likewise, when the dapsone 5% gel is administered twice daily, it can be done at various times such as once in the morning and once at night each day.

In some embodiments, the dapsone 5% gel can be administered for a period of time effective to improve the acne vulgaris. In some embodiments, the period of time effective to improve the acne vulgaris can be about 12 weeks. The period of time effective to improve the acne vulgaris can be about 4 weeks, about 8 weeks, about 10 weeks, about 16 weeks, about 20 weeks, and the like. According to some embodiments, the period of time effective to improve the acne vulgaris can be about 12 weeks or more, about 10 weeks or more, about 8 weeks or more, about 4 weeks or more, and the like. According to some embodiments, the period of time effective to improve the acne vulgaris can be determined by a patient's physician.

In some embodiments, an improvement in acne vulgaris can include a reduction in the severity of a patient's acne vulgaris. For example, an improvement in acne vulgaris can, for example, include a reduction in the number of inflammatory and/or non-inflammatory lesions, comedones, papules/pustules or nodulocystic lesions present on the face of the patient with acne vulgaris. In the some embodiments, improvement can be present where a patient's nodules change from inflammatory to non-inflammatory. According to some embodiments, an improvement in acne vulgaris can include a reduction of the acne vulgaris to clear (e.g. no or nearly no evidence of acne vulgaris) or almost clear (e.g. rare non-inflammatory lesions present, with rare non-inflamed papules) as assessed by a physician and/or self-assessed by the patient.

According to some embodiments, the improvement in acne vulgaris is greater in adult female patients compared to adolescent female patients. In one embodiment, the treatment results in statistically greater significant reductions in non-inflammatory and total lesion counts in the adult female compared to adolescent females (aged 12-17 years), wherein the reduction in inflammatory lesion is statistically the same in both the adult and adolescent female. For example, according to methods of treatment disclosed herein, the reduction in non-inflammatory lesion count can be about 20-33% higher (e.g., 20% higher, 25% higher, 30% higher, 33% higher) in the adult female compared to the reduction in the adolescent female. . In another embodiment, the reduction in total lesion count is about 15-20% higher (e.g., 15% higher, 17% higher, or 20% higher) in adult females compared to the reduction in adolescent females.

### EXAMPLE

Subgroup analysis of female subjects with AV receiving active treatment enrolled in 2 randomized double-blind clinical trials was conducted to determine whether the response to dapsone 5% gel was similar in adolescent girls (age 12-17 years) and adult women (age greater than or equal to 18 years) with facial acne vulgaris (AV).

This subgroup analysis was based on data from two identically designed 12-week, multicenter, randomized, double-blind phase III studies of dapsone 5% gel BID versus vehicle gel (n=3010), as described previously. Subjects (≧12 years of age) were randomly assigned to either treatment and instructed to apply the test material twice daily to the face after washing with a standardized soap-free cleanser. Subjects included in the trials had clinically diagnosed AV, with 20-50 inflammatory lesions and 20-100 non-inflammatory lesions located on the face above the mandibular line at baseline. Subjects were excluded if severe cystic acne or conglobate acne was present or if any emerging nodular lesions above the mandible were noted. Subjects were also excluded if they used other treatments (i.e. topical, systemic, devices) that could affect AV, including the use within 4 weeks of baseline of any systemic or immunosuppressive agents or other therapies known to affect AV or inflammatory responses and/or the use of oral isotretinoin within 3 months of baseline.

Study Visits and Assessments. Enrolled subjects were evaluated at baseline and at weeks 2, 4, 6, 8, and 12. At each visit, all subjects underwent investigator global assessment using the Global Acne Assessment Score (GAAS) which was recorded, and counting of inflammatory and non-inflammatory AV lesions which were recorded, along with the total AV lesion count. The GAAS was scored on a 5-point scale (0=none, 1=minimal, 2=mild, 3=moderate, and 4=severe).

Efficacy Assessments

Efficacy at the 12 week time point was assessed by comparing mean GAAS score at baseline and endpoint as well as change from baseline. In addition, efficacy was evaluated based on the proportion of subjects achieving success on the GAAS, defined as achieving a rating of none (0) or minimal (1). Efficacy was also determined via acne lesion counts. Endpoint success for AV lesions was defined as a significantly greater mean percentage reduction from baseline in at least two of the three types of AV lesions (inflammatory, non-inflammatory, total) at week 12.

Tolerability and Safety Monitoring. Adverse events (AEs), including signs and symptoms of application site reactions (local skin tolerability), were monitored and captured throughout the study at each study visit or if reported otherwise by the study subject.

Statistical Methods

Within-group change from baseline was analyzed and comparisons were performed using the Wilcoxon signed-rank test. Ranked analysis of covariance was used to analyze the percentage of reduction in acne lesion counts

Results

Subject Disposition and Characteristics.

Among the 2507 evaluable subjects who completed the two phase III pivotal trials, there were 781 female subjects in the dapsone 5% gel group, (347 adolescent girls 12-17 years of age and 434 adult women >18 years of age) who were eligible for this analysis (Table 1). The mean age of the adolescent subgroup was 14 years and the mean age of the adult subgroup was 27 years. Most subjects were White, with the race/ethnicity definitions and their dispositions outlined in Table 1. At baseline, there was no difference between groups in GAAS scores. The mean numbers of AV lesions were higher at baseline in the adolescent subgroup by 1.5 inflammatory lesions, 7.8 non-inflammatory lesions, and 9.2 total lesions, however, the standard deviation ranges indicate that there was considerable overlap in lesion types and quantities between both age-related female subgroups.

Efficacy

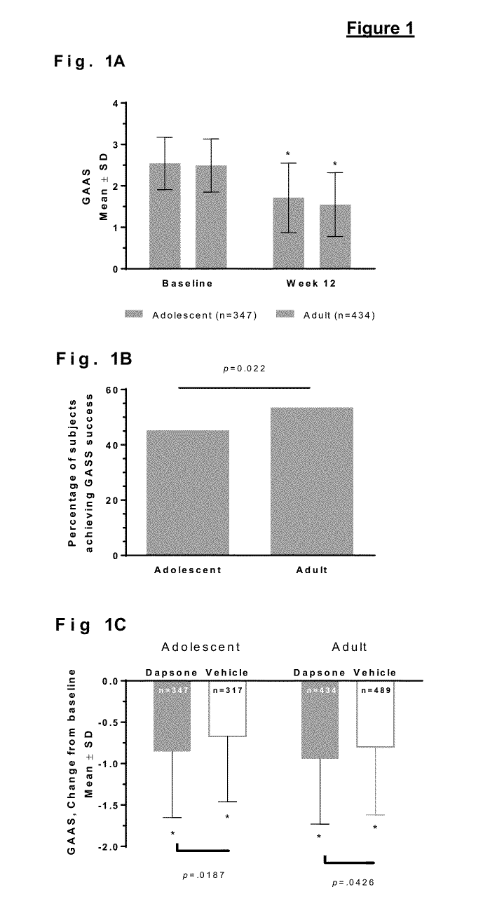

At week 12, dapsone 5% gel improved AV in both the adolescent and adult female subgroups, as demonstrated by significantly reduced mean GAAS in both subsets (p<0.001) (FIG. 1A). However, the proportion of subjects with clinical success (score 0 or 1 on GAAS) at week 12 was greater in adult women (53.5%) versus adolescents(45.3%, p=0.022; FIG. 1B). Dapsone 5% gel significantly reduced mean GAAS from baseline (p<0.001) in both groups, with no differences in mean change from baseline to week 12 in GAAS between dapsone-treated adolescent girls and adult women FIG. 1C). Both groups showed significantly greater mean changes from baseline in GAAS than their vehicle treated counterparts: for adolescents, dapsone 5%: −0.85, vehicle: −0.67 (p=0.0187), for adults dapsone 5%: −0.94, vehicle: −0.80 (p=0.0426).

Treatment with dapsone 5% gel resulted in statistically significant reductions in inflammatory, non-inflammatory, and total lesion counts expressed as percentage change from baseline in both adolescent girls and adult women (Table 2). The percentage changes from baseline in all lesion endpoints were numerically greater for each type of lesion in adult women compared to adolescent girls. In the dapsone 5% gel group, statistically significant reductions in adults were greater compared with adolescent girls, and were observed for both non-inflammatory (p<0.0001) and total lesion counts (p=0.0008).

Tolerability and Safety

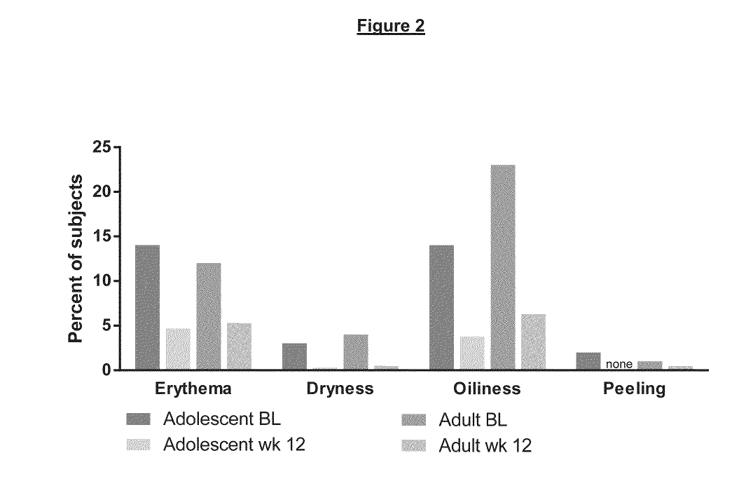

Facial application of dapsone 5% gel for 12 weeks reduced (improved) the percentage of subjects reporting erythema, dryness, oiliness, and peeling compared with baseline in both adolescent girls and adult women (FIG. 2). This current analysis of AE data specifically from both female subgroups showed no major safety issues and there were no previously unrecognized safety signals.

Discussion

The subgroup analysis reported here was designed to compare the efficacy of dapsone 5% gel applied BID in two subgroups of female patients based on age: adolescent girls (age 12-17 years) and adult women (age >18 years). Our results demonstrated that dapsone 5% gel was effective in both adolescent and adult females in reducing facial AV. A comparable reduction was observed in inflammatory AV lesions in both adult and adolescent females, while reductions in non-inflammatory and total AV lesions were greater in adult women as compared with adolescents. These data suggest that the efficacy of dapsone 5% gel applied twice a day for facial AV, although shown to be effective in females >12 years of age, may exhibit greater efficacy overall in adult women (>18 years of age) as compared with adolescent females (age 12-17 years).

GAAS responder rates in both adolescent (45.3%) and adult females (53.5%) were greater than the overall responder rate (40.5%) reported from the combined male and female population pivotal phase III studies, confirming that dapsone 5% gel was more effective in females overall. Dapsone 5% gel was equally effective in both age groups in reducing the percentage change from baseline in inflammatory lesions. Interestingly, although adolescent girls had significantly greater numbers of both inflammatory and non-inflammatory AV lesions at baseline than did adult women, dapsone 5% gel was more effective in reducing non-inflammatory lesions in adult women as compared to reductions noted in adolescents.

A total of 347 adolescent and 434 adult women were included in the subgroup analysis. At week 12, dapsone 5% gel significantly reduced mean GAAS in both subgroups (p<0.001); however, surprisingly the proportion of subjects with clinical success (no or minimal acne based on GASS) at week 12 was greater in adult (53.5%) versus adolescent females (45.3%, p=0.022). Significantly greater percentage reductions in both non-inflammatory (p<0.0001) and total lesion counts (p=0.0008) were observed in adult females compared with adolescent females. No major safety issues and no previously unrecognized safety signals were noted.

The subgroup analysis data reported here, based on female patients actively treated with dapsone 5% gel BID, support that this agent is effective, well tolerated, and safe in both adolescent and adult women, and suggest that efficacy may be greater in the latter subgroup.

TABLE 1
Demographics and Clinical Characteristics at Baseline

| | Adolescent (<18 y) | Adult (≧18 y) | |
|---|---|---|---|
| Characteristic | N = 347 | N = 434 | p Value |
| Age, y, mean ± SD | 14.4 ± 1.6 | 27.4 ± 7.8 | <.001 |
| (range) | (12-17) | (18-63) | |
| Race/Ethnicity, n (%) | | | |
| White^{a} | 266 (76.7) | 267 (61.5) | .0001 |
| Black | 50 (14.4) | 106 (24.4) | |
| Hispanic | 27 (7.8) | 42 (9.7) | |
| Asian | 1 (0.3) | 13 (3.0) | |
| Multiracial^{b} | 1 (0.3) | 3 (0.7) | |
| Other^{c} | 2 (0.6) | 3 (0.7) | |
| GAAS | 2.5 ± 0.6 | 2.5 ± 0.6 | .2 |
| Inflammatory lesions | 29.0 ± 8.9 | 27.5 ± 8.1 | .0233 |
| Non-inflammatory | 50.1 ± 22.5 | 42.3 ± 23.4 | <.0001 |
| lesions | | | |
| Total lesions | 79.0 ± 26.6 | 69.8 ± 25.9 | <.0001 |

Data are reported as mean ± SD unless otherwise indicated.
^{a}Includes Arabic, Armenian, eastern European, Egyptian, Palestinian, South American, Turkish, and White.
^{b}Includes bi-racial, Black/White, and Black/Hispanic.
^{c}Includes Brazilian, Indian, Métis, Native American, Asian Indian, East Indian, and Mulatto.
GAAS = Global Acne Assessment Scale.

TABLE 2
Percentage Change From Baseline in Lesion Counts at Week 12

| Lesion Type, | | Adolescent | Adult | |
|---|---|---|---|---|
| (Mean ± SD) | Treatment | (<18 y) | (≧18 y) | p-Value^{a} |
| Inflammatory | Dapsone | −56.2 ± 33.3 | −60.0 ± 33.3 | .3 |
| lesions | Vehicle | −48.0 ± 40.6 | −56.8 ± 34.7 | |
| Non-inflammatory | Dapsone | −35.5 ± 41.8 | −47.4 ± 38.6 | <.0001 |
| lesions | Vehicle | −23.5 ± 48.8 | −42.3 ± 35.6 | |
| Total lesions | Dapsone | −44.0 ± 32.7 | −52.8 ± 29.7 | .0008 |
| | Vehicle | −33.7 ± 35.8 | −48.3 ± 30.3 | |

^{a}Comparison between age groups.
Adolescents: Dapsone treated, n = 347, vehicle treated, n = 317
Adults: Dapsone treated, n = 434, vehicle treated, n = 489

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the relevant art and are intended to fall within the scope of the appended claims.

What is claimed is:

1. A method of treating facial acne vulgaris in an adult (≧18 years of age) female in need of such treatment, comprising topically administering dapsone 5 wt % gel twice daily to the face of the adult female.

2. The method of claim 1, wherein the treatment results in statistically greater significant reductions in non-inflammatory and total lesion counts in the adult female compared to an adolescent (aged 12-17 years) female, and wherein reduction in inflammatory lesion count is statistically the same in both the adult female and adolescent female.

3. The method of claim 2, wherein the reduction in non-inflammatory lesion count is about 20% to about 33% higher in the adult female compared to the reduction in non-inflammatory lesion count in the adolescent female.

4. The method of claim 2, wherein the reduction in total lesion count is about 15% to about 20% higher in the adult female compared to the reduction in total lesion count in the adolescent female.

5. The method of claim 1, wherein the treatment is for a period of 12 weeks.

6. A method of reducing the non-inflammatory and total lesion counts associated with acne vulgaris in an adult (≧18 years of age) female, the method comprising topically administering dapsone 5 wt % gel twice daily to the face of the adult female.

7. The method of claim 6, wherein the treatment results in a statistically significant reduction in inflammatory lesion count in the adult female that is statistically the same as the reduction in inflammatory lesion count in an adolescent (aged 12-17 years) female.

8. The method of claim 7, wherein the reduction in non-inflammatory lesion count is about 20% to about 33% higher in the adult female compared to the reduction in non-inflammatory lesion count in the adolescent female.

9. The method of claim 7, wherein the reduction in total lesion count is about 15-20% higher in the adult female compared to the reduction in total lesion count in the adolescent female.

10. The method of claim 6, wherein the treatment is for a period of about 12 weeks.

11. A method of increasing the efficacy of dapsone 5 wt % gel in treating acne vulgaris in an adult female population, comprising topically administering dapsone 5 wt % gel twice daily to the face of adult (≧18 years of age) females in need thereof, wherein said administering results in statistically greater significant reductions in non-inflammatory and total lesion counts in the adult females compared to adolescent (aged 12-17 years) females, thereby increasing the efficacy of dapsone 5 wt % gel in treating acne vulgaris in the adult female population.

12. The method of claim 11, wherein the reduction in inflammatory lesion count is statistically the same in both the adult females and adolescent females.

13. The method of claim 11, wherein the reduction in non-inflammatory lesion count is about 20% to about 33% higher in the adult females, compared to the non-inflammatory lesion count reduction in the adolescent females.

14. The method of claim 11, wherein the reduction in total lesion count is about 15% to about 20% higher in the adult females compared to the reduction in total lesion count of the adolescent females.

15. The method of claim 11, wherein said administering is for a period of about 12 weeks.
